(19) United States
(12) Patent Application Publication
(10) Pub. No.: US 2022/0304986 A1
(43) Pub. Date: Sep. 29, 2022

(54) COMPOSITIONS AND METHODS FOR TREATING ACQUIRED BRAIN INJURY AND POST-TRAUMATIC STRESS DISORDER

(71) Applicant: Thomas Winston, Stilwell, KS (US)

(72) Inventor: Thomas Winston, Stilwell, KS (US)

(21) Appl. No.: 17/656,855

(22) Filed: Mar. 28, 2022

Related U.S. Application Data
(60) Provisional application No. 63/166,752, filed on Mar. 26, 2021.

(51) Int. Cl. A61K 31/436; A61K 31/23; A61K 31/155; A61K 31/352; A61K 31/593; A61K 31/07; A61K 31/519; A61K 31/375; A61K 31/355; A61K 31/122; A61K 31/51; A61K 31/525; A61K 31/455; A61K 31/4188; A61K 31/197; A61K 33/06; A61K 33/42; A61K 33/18; A61K 33/30; A61K 33/34; A61K 33/04; A61K 33/32; A61K 33/24

(52) U.S. Cl. CPC A61K 31/436; A61K 31/23; A61K 31/155; A61K 31/352; A61K 31/593; A61K 31/07; A61K 31/519; A61K 31/375; A61K 31/355; A61K 31/122; A61K 31/51; A61K 31/525; A61K 31/455; A61K 31/4188; A61K 31/197; A61K 33/06; A61K 33/42; A61K 33/18; A61K 33/30; A61K 33/34; A61K 33/04; A61K 33/32; A61K 33/24

(57) ABSTRACT

Disclosed herein are pharmaceutical compositions for the treatment of acquired brain injury (ABI), including traumatic brain injury (TBI) and acute non-traumatic brain injury (ANBI), post-traumatic stress disorder (PTSD), and other degenerative neurological disorders that may or may not be associated with ABI comprising an effective amount of one or more mTOR inhibitors and optionally an effective amount of one or more thyroid hormones. Further disclosed herein are methods of treating, preventing, reducing the intensity of or reducing the severity of ABI and/or PTSD, the method comprising administering an effective amount of one or more mTOR inhibitors and optionally an effective amount of one or more thyroid hormones.

### FIELD

The field of the disclosure relates generally to compositions for the treatment of acquired brain injury (ABI), including traumatic brain injury (TBI) and acute non-traumatic brain injury (ANBI), post-traumatic stress disorder (PTSD) that may or may not be associated with ABI, Alzheimer's Disease, Parkinson's Disease, Amyotrophic lateral sclerosis (ALS), Friedreich ataxia, Huntington's disease, Lewy body disease, Spinal muscular atrophy, Supranuclear Palsy, and other degenerative neurologic disorders—(hereinafter “ABI, TBI, PTSD, and the disorders”). More specifically, the field of disclosure relates generally to compositions for the treatment of ABI, TBI, PTSD, and the disorders that include one or more mammalian target of rapamycin (mTOR) inhibitors optionally combined with thyroid hormones.

More specifically, the field of disclosure relates to treating ABI, TBI, PTSD, and the disorders including closed head injuries and penetrating head injuries as a result of one or more physical traumas, ANBI, including those derived from either internal or external sources, as well as the mental condition, PTSD, whether or not it is associated with TBI resulting from closed or penetrating head injuries, ANBI resulting from either internal or external sources, Alzheimer's Disease, Parkinson's Disease, Amyotrophic lateral sclerosis (ALS), Friedreich ataxia, Huntington's disease, Lewy body disease, Spinal muscular atrophy, and Supranuclear Palsy. More specifically, the field of disclosure relates to treating ABI resulting from traumatic impacts to the head and those resulting from traumatic inertial forces that affect the brain including falls, accidents, sports activities, strikes by objects, explosions and other traumatic events; as well as treating ABI resulting from non-traumatic causes including stroke, anoxia, hypoxia, toxins, infections, cancer and other internal and external factors; treating the mental condition, PTSD, that can result from exposure to physical or emotional trauma or abuse, stressful experiences, mental illnesses, witnessing horrific events, exposure to toxins, substance abuse and other internal and external factors; and treating other degenerative neurological disorders.

### BACKGROUND

Acquired brain injury (ABI) includes injury to the brain that is not hereditary or congenital and results in abnormal brain function from changes in neuronal integrity or activity. Traumatic and acute non-traumatic forms of ABI can result from a wide range of forces and factors that affect the brain including falls, accidents, sports activities, strikes by objects, explosions and other traumatic events as well as stroke, anoxia, hypoxia, toxins, infections, cancer and other internal and external factors. Treatment of ABI is based on the severity of the injury and can range from no treatment, or simple rest with monitoring, to immediate emergency and follow up surgical care to maintain adequate blood pressure, blood supply and oxygen to the brain. Treatment may also include rehabilitative therapy and providing medications to limit secondary damage to the brain due to inflammation, cellular instability, hemorrhage, and reduced oxygen supply. Medications that are often used to limit secondary damage immediately after an injury include anti-inflammatory drugs, diuretics, anticoagulants, anticonvulsants, muscle relaxants and coma-inducing drugs. Regardless of the severity of the injury, inflammation and cell integrity are considered important factors in the pathophysiology of ABI. However, despite much research that indicates certain anti-inflammatory and neuroprotective drugs dosed immediately before or soon after ABI may have a significant therapeutic effect, increasing the time interval between injury and first dose to clinically relevant time periods often greatly lowers efficacy. New treatment approaches are needed.

Post-traumatic stress disorder (PTSD) is a mental condition or disorder that can result from exposure to physical or emotional trauma or abuse, stressful experiences, mental illnesses, witnessing horrific events, exposure to toxins, substance abuse and other internal and external factors. The main treatments for PTSD include psychological therapies and medicines that include selective serotonin reuptake inhibitor (S SRI) and serotonin-norepinephrine reuptake inhibitor (SNRI) antidepressants that reduce the symptoms of the disorder and are effective only for a fraction of patients. New treatment approaches are needed.

Degenerative neurological disorders such as Alzheimer's Disease, Parkinson's Disease, Amyotrophic lateral sclerosis (ALS), Friedreich ataxia, Huntington's disease, Lewy body disease, Spinal muscular atrophy, Supranuclear Palsy are typified by a decline in function over time. There are no current treatments for these disorders and therefore treatment approaches are needed.

### BRIEF DESCRIPTION

Disclosed herein is a pharmaceutical composition for the treatment of ABI, TBI, PTSD, and/or the other disorders including an effective amount of one or more mTOR inhibitors and optionally an effective amount of one or more thyroid hormones.

In another aspect, disclosed herein is a method of treating or preventing ABI or TBI in a patient, including administering an effective amount of a pharmaceutical composition including one or more mTOR inhibitors and optionally an effective amount of one or more thyroid hormones.

In another aspect, disclosed herein is a method of treating or preventing PTSD in a patient, including administering an effective amount of a pharmaceutical composition including one or more mTOR inhibitors and optionally an effective amount of one or more thyroid hormones.

In another aspect, disclosed herein is a method of treating or preventing at least one disorder selected from the group consisting of Alzheimer's Disease, Parkinson's Disease, Amyotrophic lateral sclerosis (ALS), Friedreich ataxia, Huntington's disease, Lewy body disease, Spinal muscular atrophy, and Supranuclear Palsy (“the disorders”), in a patient, including administering an effective amount of a pharmaceutical composition including one or more mTOR inhibitors and optionally an effective amount of one or more thyroid hormones.

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “patient” refers to a warm: blooded animal such as a mammal which is the subject of a medical treatment for a medical condition that causes at least one symptom. It is understood that at least humans, dogs, cats, and horses are within the scope of the meaning of the term. In some aspects, the patient is human. Generally, as used herein, the term “patient” means a human or an animal for which the compositions of the disclosure may be administered.

As used herein, the terms “treat”, “treating”, and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms encompass also therapy and cure. Treatment means any manner in which the symptoms of a pathological condition, disorder, or disease are ameliorated or otherwise beneficially altered.

As used herein, the term “preventing” includes administration of a composition which reduces the frequency of, or delays the onset of, or symptoms of a medical condition in a subject relative to a subject which does not receive the composition.

As used herein, the term “reduce the incidence of” refers to a reduction in the number of clinical signs or symptoms of a medical condition in a subject that is administered a composition relative to a subject which does not receive the composition.

As used herein, the term “reduce the severity of” refers to a reduction in the severity of clinical signs or symptoms of a medical condition in a subject that is administered a composition relative to a subject which does not receive the composition.

As used herein, the term “acquired brain injury (ABI)” refers to disorders of the brain that are caused by injury to the brain that is not hereditary or congenital and results in abnormal brain function from changes in neuronal integrity or activity. ABI includes both traumatic and acute non-traumatic forms of ABI that can result from a wide range of forces and factors that affect the brain. The primary injury can result from traumatic impact that is either skull penetrating or non-penetrating, from traumatic inertial forces causing brain movement within the skull, or from non-traumatic internal or external injurious factors. Examples of ABI include disorders resulting from falls, accidents, assaults, sports activities, strikes by objects, stabbings, gunshots, explosions and other traumatic events as well as stroke, anoxia, hypoxia, ischemia, toxins, infections, tumors and other internal and external factors. Additional examples of ABI include brain contusions, brain lacerations, intracerebral hemorrhage, diffuse axonal injury, epidural hematomas, subdural hematomas, white matter lesions, hemorrhage, severe concussion, and other ABI pathophysiology.

As used herein, the term “post-traumatic stress disorder (PTSD)” refers to a mental condition or disorder that can result from causes including exposure to physical or emotional trauma or abuse, stressful experiences, mental illnesses, witnessing horrific events, exposure to toxins, substance abuse and other internal and external factors. Examples of symptoms of PTSD include the avoidance of trauma-related thoughts and emotions, the avoidance of discussion or amnesia associated with the injurious experience, reliving or recollecting the injurious experience in flashbacks and nightmares and similar experiences that generally are sufficiently persistent beyond one month.

As used herein, the term “mTOR complex 1 (mTORC1)” refers to a protein complex comprising mTOR, regulatory-associated protein of mTOR (RAPTOR), mammalian lethal with SEC13 protein 8 (mLST8), proline-rich AKT substrate of 40 kDa (PRAS40) and DEP domain-containing protein 6 (DEPTOR) that has been described to function as a nutrient/energy/redox sensor; regulator of cellular growth, proliferation, and motility; and controller of protein synthesis with roles in inflammation, autophagy and cell survival.

As used herein, the term “mTOR complex 2 (mTORC2)” refers to a protein complex comprising mTOR, mLST8, DEPTOR, rapamycin-insensitive companion of mTOR (RICTOR), mammalian stress-activated protein kinase interacting protein 1 (mSIN1), and protein observed with rictor 1 and 2 (PROTOR1/2) that has been described to function as an activator of insulin receptors and insulin-like growth hormone factor 1 receptors; and regulator of cell proliferation, cell migration and cytoskeletal remodeling with roles in signaling the production of cytokines, inflammation and cell survival.

As used herein, the term “mTOR inhibitor (mTOR Inhibitor)” refers to a composition that either directly or indirectly inhibits one or more functions of at least one of mTOR, mTORC1, mTORC2 and combinations thereof. Examples of suitable mTOR inhibitors include omega-3 fatty acid derivatives, biguanide antihyperglycemic agents, flavonoids, non-antibiotic macrolides, and other agents that effectively inhibit one or more mTOR protein complex functions.

As used herein, the term “thyroid hormone” refers to a composition that is either equivalent to, a derivative of, or affects the same functions as triiodothyronine (T3). Examples of suitable thyroid hormones include liothyronine, a T3 thyroid hormone composition.

Without being bound by theory, it is believed that the pathology of ABI, TBI, PTSD, and the disorders involve an underlying excessive inflammation caused by senescent cells in the brain and the rest of the body as well as the effects of inflammatory cytokines and other chemicals.

Without being bound by theory, it is also believed that the makeup of the neuronal and mitochondrial membranes may be key elements for the maintenance of cellular function and architecture. It is believed that abnormalities of the membrane may degrade cellular function over time and ultimately cause the death of the cells. Part of the maintenance of the cell membrane may involve maintaining an optimal ratio of omega-3 fatty acids to omega-6 fatty acids in the cellular membrane, which results in an anti-inflammatory effect. It is believed that, over time, with oxidative stress, changes within the genes, such as mTOR gene regulation, may lead to degradation of the omega-6 to omega-3 fatty acid ratio.

Without being bound by theory, it is also believed that mTORC1 and mTORC2 control multiple diffuse aspects of cellular metabolism, cellular integrity, cellular death and inflammation. It is believed that the mTORC1 and mTORC2 activity is enhanced and driven upwards by cytokine release including those released as a result of higher than optimal ratios of omega-6 to omega-3 fatty acids in the cell membrane and that mTORC1 and mTORC2 complex functions may be down regulated by the use of mTOR inhibitors. Examples of suitable mTOR inhibitors may include omega-3 fatty acid derivatives, biguanide antihyperglycemic agents, flavonoids, non-antibiotic macrolides, and other agents that effectively inhibit mTOR protein complexes.

Examples of omega-3 fatty acids include eicosapentaenoic acid (EPA) and docosahexaenoic acid (DHA), which are generally derived from diet. It is believed that EPA is superior to DHA for inhibition of inflammation and maintaining cell integrity. It is believed that omega-6 fatty acids (e.g. arachidonic acids) are precursors to the formation of cytokines. It is believed that omega-3 fatty acids may help to decrease cytokine production, for example, from the action of eicosanoid molecules. Accordingly, a decrease of omega-3 fatty acids, in relation to omega-6 fatty acids, may facilitate an inflammatory response caused by cytokines. An increase in cytokines may not only promote excessive inflammation, but also cause damage to cell synapses and ultimately may lead to cellular death.

Without being bound by theory, it is believed that omega-3 fatty acids may downshift cellular signaling by decreasing cytokine formation. This may increase the maintenance of cellular adhesion and normal membrane anatomy with better sodium, potassium and calcium channel function and better response to stimuli from hormones, cell signaling proteins and other cell signaling substances such as nitric oxide. Therapies including omega-3 fatty acids may also facilitate the maintenance of membranes of mitochondria and other intracellular structures.

It is further believed that control over mTOR-associated protein complexes may also be of importance in the prevention, reduction in incidence of and reduction in severity of ABI, TBI, PTSD, and/or the disorders. It is believed that mTOR-associated protein complexes may respond to stimuli that alter cellular metabolism and growth. The mTOR-associated protein complexes may be involved in many diseases and almost all tissues of the body, including the brain. It is believed that the dysregulation of mTORC1 and mTORC2 may be an underlying cause of disease over one's lifetime. It is also believed that overactivity of these protein complexes may lead to a higher incidence and severity of secondary damage to the brain associated with ABI and/or PTSD due to inflammation and/or cellular instability.

Without being bound by theory, it is believed that mTOR-associated protein complexes are critical in the regulation of metabolism and inflammation in key metabolic tissues including the brain. It is further believed that biguanide antihyperglycemic agents act through inhibition of the mTORC2 complex to modulate cell functions including metabolism, proliferation, migration and survival as well as reduce oxidative stress and inflammation. It is also believed that biguanide antihyperglycemic agents inhibit the mTORC2 complex by mechanisms including the reduction of the downstream effects of the AKT protein that is a component of the PI3K/AKT/mTOR pathway. By modulating cell functions and reducing oxidative stress and inflammation through its reduction of the downstream effects of the PI3K/AKT/mTOR pathway, biguanide antihyperglycemic agents may be effective in treating, preventing, reducing the incidence of or reducing the severity of ABI, TBI, PTSD, and/or the disorders and/or PTSD.

Without being bound by theory, it is believed that certain flavonoids act as senolytic agents by reducing mTOR complex activity, increasing the activity of sirtuins, and increasing the activity of AMP-activated protein kinase (AMPK). These actions are believed to play a role in cellular energy homeostasis and promotion of apoptosis in senescent cells that are resistant to signaling proteins and accumulate during the aging process as we age. It is further believed that the accumulation of senescent cells results from a weakened immune system related to aging, and these cells provide a source of chronic inflammation through the release of inflammatory chemicals and may lead to an increased risk of cancer. It is also believed that the mechanistic actions of certain flavonoids used in combination with a biguanide antihyperglycemic agent can exhibit synergistic effects for promoting apoptosis in senescent cells while promoting homeostasis in normal cells. It is further believed that when certain flavonoids are combined with certain galactomannans, the absorption of the certain flavonoids can be increased by as much as 25-fold.

Without being bound by theory, it is believed that certain non-antibiotic macrolides act directly as mTOR inhibitors and will facilitate the treatment of ABI and/or PTSD either alone or in combination with other mTOR inhibitors. It is also believed that certain non-antibiotic macrolides may facilitate the rapid treatment of ABI and/or PTSD by emergency healthcare professionals, first responders and soldiers away from a traditional treatment center, by administration either orally or intravenously as soon as possible following diagnosis. Examples of suitable non-antibiotic macrolides includes rapamycin and rapamycin derivatives. It is further believed that the effectiveness of ABI treatment with certain non-antibiotic macrolides may be further enhanced when combined with a corticosteroid to facilitate a decrease in brain inflammation and swelling.

Thyroid hormones, including e.g. liothyronine (a T3 thyroid hormone), are believed to assist in controlling metabolism by utilizing oxygen and calories for conversion into energy in the mitochondria through the formation of ATP. Thyroid hormones are believed to be necessary for energy production in all organs, especially in muscle, brain, heart, and other tissues. Increased levels of thyroid hormones are believed to affect increased levels of cellular metabolism. Various tests are available to determine thyroid hormone levels, e.g. by measuring the amount of thyroid hormone levels in the blood. Thyroid hormones are believed to enhance cell survival and the metabolism of fats, proteins and carbohydrates. It is further believed that treatment comprising one or more thyroid hormones in combination with a flavonoid, such as fisetin may act synergistically to increase metabolism and promote the senolytic effects of fisetin.

Thyroid hormones are believed to affect nearly every cell of the body through receptors in the nucleus of the cell. Thyroid hormones bind to DNA-binding nuclear hormone receptors, cause conformational changes in the receptors, and activate transcription of the thyroid hormone sensitive genes by either initiating expression or upregulation. Also, functions of the PI3K/AKT pathway are believed to include regulation of cell adhesion, cell cycle progression, cell survival and signaling. Precursors to the thyroid hormones, referred to as T4 or thyroxine, are believed to stimulate the PI3/AKT pathway in the cytoplasm, whereas T3 does not. T3 also has a shorter half-life than T4, so T3 is recommended for the treatment of hepatic steatosis over T4.

Without being bound by theory, it is believed that rapamycin is primarily an mTOR1 inhibitor at lower doses and for short treatment cycles, whereas high levels and very prolonged treatment cycles can also inhibit mTOR2 by blocking mTOR2 production by the cell. Rapamycin treatment is normally administered continuously either orally or intravenously, which frequently causes side effects of insulin resistance and hyperglycemia, and causes immune deficiency. Also, long-term treatment with rapamycin may decrease antigen processing and inhibit T-cell proliferation leading to suppression of the immune system. Rapamycin is also believed to decrease the phosphorylation of the ribosomal s6 kinase, S6K1, which is believed to result in active decreases in protein synthesis and cell mortality.

It is believed that treatment regimens that included rapamycin could be effective and safe if rapamycin is dosed at low-levels either intermittently or in conjunction with other mTOR inhibitors and/or additional medications that decrease or down regulate the PI3K-AKT pathway. It is further believed that using a biguanide antihyperglycemic agent, such as metformin, in these treatment regimens will allow for down regulation of both mTOR1 and mTOR2 safely without causing significant side effects of high-dose rapamycin. In addition to acting as an inhibitor of mTOR2, metformin also decreases glycolysis and is effective in controlling blood glucose levels. The addition of a flavonoid, such as fisetin, is believed to provide the added benefit of promoting apoptosis or cell death of senolytic cells effectuated at least partly through its inhibition of the mTOR pathway. The effects of fisetin may be further improved with the addition of a T3 thyroid hormone. It is believed that synergy of activity for inhibition of the PI3K-AKT pathway can be achieved with the combination of rapamycin, metformin, and fisetin while providing a low risk of side effects. The treatment regimens could further benefit from the addition of an omega-3 fatty acid derivative, which is believed to downregulate mTOR2, decrease cytokine formation, strengthen cell membranes and structures, and decrease phosphorylation of phosphatides. Additionally, the addition of a T3 thyroid hormone is believed to enhance the effectiveness of the therapy regimen. These combination therapies are believed to have minimal side effects, may be administered continuously over long periods of time, and result in an effective decrease in PI3K-AKT activity.

In various embodiments, the compositions of the disclosure include compositions for the treatment of ABI and/or PTSD In various embodiments, the compositions of the disclosure include an effective amount of one or more mTOR inhibitors and optionally an effective amount of one or more thyroid hormones. In various embodiments, suitable mTOR inhibitors may include omega-3 fatty acid derivatives, biguanide antihyperglycemic agents, flavonoids, non-antibiotic macrolides, and other agents that effectively inhibit mTOR protein complexes. In various embodiments, suitable thyroid hormones may include a T3 hormone, such as liothyronine.

Preferentially, at least one of the components of the composition will decrease excessive inflammation and swelling associated with ABI, TBI, PTSD, and/or the disorders.

Preferentially, at least one of the components of the composition will enhance neuronal cell integrity and function and/or induce apoptosis in senescent cells. Preferentially, the compositions of the disclosure include a flavonoid, such as fisetin, at doses that are high enough to cause senescent cells to die and results in an overall decrease in inflammation in the patient.

Preferentially, the compositions of the disclosure include at least an effective amount of a biguanide antihyperglycemic agent in combination with an effective amount of an omega-3 fatty acid derivative.

Preferentially, other than promoting euthyroid in patients, the compositions of the disclosure include a thyroid hormone concurrent with high doses of a flavonoid, such as fisetin.

Preferentially, at least one of the components of the composition will decrease the rate of neuronal cell death or will increase the life span of neuronal cells.

In various embodiments, the compositions may include an effective amount of an omega-3 fatty acid derivative. Suitable omega-3 fatty acid derivatives may include icosapent ethyl. In various embodiments, the compositions of the disclosure may include an effective amount of at least about 0.5 g of icosapent ethyl, or between about 0.5 g to about 8.0 g of icosapent ethyl, or 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 g, or any range between any two of these amounts including about 0.5 g to about 8.0 g, or about 1.0 g to about 7.0 g, or about 2.0 g to about 8.0 g of icosapent ethyl. In some preferred forms, the amount of icosapent ethyl is sufficient to maintain an optimal level of icosapent ethyl in the blood of a subject receiving an administration of the composition.

In various embodiments, the compositions of the disclosure may include an effective amount of a biguanide antihyperglycemic agent. Suitable biguanide antihyperglycemic agents include metformin. In various embodiments, the compositions may include an effective amount of at least about 50 mg of biguanide antihyperglycemic agent, or between about 50 mg to about 3000 mg of biguanide antihyperglycemic agent, or 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, or 3000 mg or any range between any two of these amounts including about 250 mg to about 3000 mg, about 250 mg to about 500 mg, about 250 mg to about 750 mg, 250 mg to about 1000 mg, about 250 mg to about 1250 mg, about 250 mg to about 1500 mg, or between about 500 mg to about 3000 mg of biguanide antihyperglycemic agent.

In various embodiments, the compositions may include an effective amount of a flavonoid. Suitable flavonoid agents include fisetin and fisetin derivatives. In various embodiments, the compositions may include an effective amount of at least about 10 mg/kg of a flavonoid, or between about 10 mg/kg to about 100 mg/kg of a flavonoid, or 10, 15, 20, 25, 30, 35, 40, 45, 50, 55, 60, 65, 70, 75, 80, 85, 90, 95, or 100 mg/kg or any range between any two of these amounts including about 10 mg/kg to about 20 mg/kg, about 15 mg/kg to about 25 mg/kg, about 20 mg/kg to about 30 mg/kg, about 25 mg/kg to about 50 mg/kg, or about 20 mg/kg to about 100 mg/kg of a flavonoid. In some preferred forms, the amount of fisetin is sufficient to maintain an optimal level of fisetin in the blood of a subject receiving an administration of the composition, wherein such optimal level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect.

In various embodiments, the compositions may include an effective amount of a flavonoid. Suitable flavonoid agents include fisetin and fisetin derivatives. In various embodiments, the compositions may include an effective amount of at least about 50 mg of a flavonoid, or between about 50 mg to about 750 mg of a flavonoid, or 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 mg or any range between any two of these amounts including about 50 mg to about 500 mg, about 100 mg to about 750 mg, about 250 mg to about 500 mg, about 250 mg to about 750 mg, about 500 mg to about 750 mg, or about 100 mg to about 500 mg of a flavonoid. In some preferred forms, the amount of fisetin is sufficient to maintain an optimal level of fisetin in the blood of a subject receiving an administration of the composition; such optimal level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect.

In various embodiments, the compositions of the disclosure may include an effective amount of a non-antibiotic macrolide. Suitable non-antibiotic macrolides include rapamycin and rapamycin derivatives, such as temsirolimus, everolimus, ridaforolimus, and the like. In various embodiments, the compositions of the disclosure include an effective amount of at least about 1 mg of a non-antibiotic macrolide, or between about 1 mg to about 40 mg of a non-antibiotic macrolide, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg or any range between any two of these amounts including between about 2 mg to about 30 mg, and between about 6 mg to about 40 mg of a non-antibiotic macrolide.

In various embodiments, the compositions may include an effective amount of a thyroid hormone. Suitable thyroid hormones include the T3 liothyronine. In various embodiments, the compositions of the disclosure may include an effective amount of at least about 1 μg of liothyronine, or between about 1 μg to about 250 μg, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 μg, or any range between any two of these amounts including about 5 μg to about 150 μg, or about 10 μg to about 100 μg, or about 10 μg to about 25 μg, or about 25 μg to about 150 μg, or about 25 μg to about 250 μg. In some preferred forms, the amount of liothyronine is sufficient to maintain an optimum level of liothyronine in the blood of a subject receiving an administration of the composition.

In various embodiments, the compositions may include an effective amount of a combination of one or more mTOR inhibitors with an effective amount of one or more thyroid hormones. In various embodiments, the compositions may include an effective amount of one or more mTOR inhibitors with an effective amount of one or more thyroid hormones and an effective amount of a flavonoid. In various embodiments, the compositions may include an effective amount of one or more mTOR inhibitors with an effective amount of one or more thyroid hormones and an effective amount of a high dose of a flavonoid, which is associated with a senolytic effect.

In various embodiments, the compositions of the disclosure may include an effective amount of a corticosteroid. Suitable corticosteroids include dexamethasone. In various embodiments, the compositions of the disclosure include an effective amount of at least about 5 mg of a corticosteroid, or between about 5 mg to about 50 mg of a corticosteroid, or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 mg or any range between any two of these amounts including between about 5 mg to about 40 mg, and about 5 mg to about 20 mg of a corticosteroid.

In various embodiments, the compositions further include an effective amount of a vitamin D derivative. Suitable vitamin D derivatives include vitamin D3 (cholecalciferol). In various embodiments, the compositions include an effective amount of at least about 500 international units (IU) of vitamin D3, or between about 500 IU to about 60000 IU of vitamin D3 daily, or 500, 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, 25000, 25500, 26000, 26500, 27000, 27500, 28000, 28500, 29000, 29500, 30000, 30500, 31000, 31500, 32000, 32500, 33000, 33500, 34000, 34500, 35000, 35500, 36000, 36500, 37000, 37500, 38000, 38500, 39000, 39500, 40000, 40500, 41000, 41500, 42000, 42500, 43000, 43500, 44000, 44500, 45000, 45500, 46000, 46500, 47000, 47500, 48000, 48500, 49000, 49500, 50000, 50500, 51000, 51500, 52000, 52500, 53000, 53500, 54000, 54500, 55000, 55500, 56000, 56500, 57000, 57500, 58000, 58500, 59000, 59500, or 60000 IU or any range between any two of these amounts including between about 2000 IU to about 30000 IU, and about 5000 IU to about 50000 IU of Vitamin D3. In some preferred forms, the amount of Vitamin D3 is sufficient to maintain an optimal level of vitamin D3 in the blood of a subject receiving an administration of the composition.

In various embodiments, the compositions further may include an effective amount of a vitamin and/or mineral supplement including one or more of a vitamin B derivative in combination with vitamin A, vitamin C, vitamin E, vitamin K, calcium, phosphorus, iodine, magnesium, zinc, selenium, copper, manganese, chromium, molybdenum, chloride, and potassium. Suitable vitamin B derivatives include thiamin (B1), riboflavin (B2), niacin (B3), pantothenic acid (B5), pyridoxine (B6), biotin (B7), folate (B9), and cyanocobalamin (B12). In various embodiments, the compositions include an effective amount of each component in relation to their percentage of daily value (DV) as recommended by the United States Food and Drug administration (FDA). In various embodiments, the compositions include an effective amount of at least about 1% to about 5000% DV of each included vitamin B derivative, an effective amount of at least about 1% to about 500% DV of vitamin A, an effective amount of at least about 1% to about 5000% DV of vitamin C, an effective amount of at least about 1% to about 500% DV of vitamin E, an effective amount of at least about 1% to about 500% DV of vitamin K, an effective amount of at least about 1% to about 500% DV of calcium, an effective amount of at least about 1% to about 500% DV of phosphorus, an effective amount of at least about 1% to about 500% DV of iodine, an effective amount of at least about 1% to about 500% DV of magnesium, an effective amount of at least about 1% to about 500% DV of zinc, an effective amount of at least about 1% to about 500% DV of selenium, an effective amount of at least about 1% to about 500% DV of copper, an effective amount of at least about 1% to about 500% DV of manganese, an effective amount of at least about 1% to about 500% DV of chromium, an effective amount of at least about 1% to about 500% DV of molybdenum, an effective amount of at least about 1% to about 500% DV of chloride, and an effective amount of at least about 1% to about 500% DV of potassium.

In various embodiments, the compositions of the disclosure may further contain additional pharmaceutically acceptable carriers. The pharmaceutical compositions may, for example, be in a form suitable for oral administration as a tablet, capsule, pill, powder, sustained release formulation, solution or suspension, in a form suitable for parenteral injection as a sterile solution, suspension, or in a form of an emulsion for topical administration as an ointment or cream, or for rectal administration as a suppository. The pharmaceutical compositions may be in unit dosage forms suitable for single administration of precise dosages. The pharmaceutical compositions may include conventional pharmaceutical carriers or excipients. In addition, the compositions may include other medicinal or pharmaceutical agents, carriers, adjuvants, and the like.

In various embodiments, the compositions may be administered to a patient through any suitable route of administration effective in delivering an amount of active agent or active agents to a patient. Suitable routes of administration include oral, parenteral, enteral, and rectal or the like.

In some forms, the composition will comprise each of the ingredients in a single administration form, such as a pill, tablet, capsule, oral solution, injection solution, infusion solution, or any of the forms described herein. In other forms, the composition will comprise a kit comprising each of the individual ingredients, together with instructions for administering each ingredient. In some forms of the kit, certain ingredients will already be combined such that one, two, three, four, or more of the components or ingredients of the composition are in a single administration form as described herein.

In various embodiments, the compositions should be prepared in a form that is stable enough to withstand a wide range of ambient environmental conditions for a reasonable amount of time to allow emergency healthcare professionals, first responders and soldiers away from a traditional treatment center to carry and safely administer, either orally or intravenously, the compositions as soon as possible following diagnosis of ABI and/or PTSD.

Various embodiments of the disclosure further relate to methods of treating ABI and/or PTSD that include administering an effective amount of one or more mTOR inhibitors and optionally an effective amount of one or more thyroid hormones. In various embodiments, suitable mTOR inhibitors may include omega-3 fatty acid derivatives, biguanide antihyperglycemic agents, flavonoids, non-antibiotic macrolides, and other agents that effectively inhibit mTOR protein complexes. In various embodiments, suitable thyroid hormones may include a T3 hormone, such as liothyronine.

In various embodiments, the methods may include administering an effective amount of an omega-3 fatty acid derivative. Suitable omega-3 fatty acid derivatives may include icosapent ethyl. In various embodiments, the methods may include administering an effective amount of at least about 0.5 g of icosapent ethyl, or between about 0.5 g to about 8.0 g of icosapent ethyl, or 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 g, or any range between any two of these amounts including about 0.5 g to about 8.0 g, or about 1.0 g to about 7.0 g, or about 2.0 g to about 8.0 g of icosapent ethyl once, twice, or three or more times daily. In some preferred forms, the amount of icosapent ethyl is sufficient to maintain an optimal level of icosapent ethyl in the blood of a subject receiving an administration of the composition.

In various embodiments, the method may include administering an effective amount of a biguanide antihyperglycemic agent. Suitable biguanide antihyperglycemic agents include metformin. In various embodiments, the methods may include administering an effective amount of at least about 50 mg of a biguanide antihyperglycemic agent, or between about 50 mg to about 3000 mg of a biguanide antihyperglycemic agent, or 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, and 3000 mg or any range between any two of these amounts including about 250 mg to about 3000 mg, about 250 mg to about 500 mg, about 250 mg to about 750 mg, 250 mg to about 1000 mg, about 250 mg to about 1250 mg, about 250 mg to about 1500 mg, or between about 500 mg to about 3000 mg of a biguanide antihyperglycemic agent once, twice, or three or more times daily.

In various embodiments, the method may include administering an effective amount of a flavonoid. Suitable flavonoid agents include fisetin and fisetin derivatives. In various embodiments, the methods may include administering an effective amount of at least about 10 mg/kg of a flavonoid, or between about 10 mg/kg to about 100 mg/kg of a flavonoid, or 10, 15, 20, 25, 30, 35, 40, 45, 50, 55, 60, 65, 70, 75, 80, 85, 90, 95, or 100 mg/kg or any range between any two of these amounts including about 10 mg/kg to about 20 mg/kg, about 15 mg/kg to about 25 mg/kg, about 20 mg/kg to about 30 mg/kg, about 25 mg/kg to about 50 mg/kg, or about 20 mg/kg to about 100 mg/kg of a flavonoid once, twice, or three or more times daily, weekly, monthly, trimonthly or intermittently with periods between administration when no flavonoid is administered. In some preferred methods, the flavonoid may be administered each day for two days on a weekly, monthly or trimonthly basis. In some preferred methods, the flavonoid may be administered each day for two days twice monthly for six months followed by administration one day per month. In some preferred methods, the amount of fisetin is sufficient to maintain an optimal level of fisetin in the blood of a subject receiving an administration of the composition, wherein such optimal level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect.

In various embodiments, the method may include administering an effective amount of a flavonoid that is administered on a daily basis. Suitable flavonoid agents include fisetin and fisetin derivatives. In various embodiments, the methods may include administering an effective amount of a flavonoid at least about 50 mg of a flavonoid, or between about 50 mg to about 750 mg of a flavonoid, or 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 mg or any range between any two of these amounts including about 50 mg to about 500 mg, about 100 mg to about 750 mg, about 250 mg to about 500 mg, about 250 mg to about 750 mg, about 500 mg to about 750 mg, or about 100 mg to about 500 mg of a flavonoid once, twice, or three or more times daily. In some preferred methods, the flavonoid may be administered each day for two days on a weekly, monthly or trimonthly basis. In some preferred methods, the flavonoid may be administered each day for two days twice monthly for six months followed by administration one day per month. In some preferred methods, the amount of fisetin is sufficient to maintain an optimal level of fisetin in the blood of a subject receiving an administration of the composition, wherein such optimal level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect.

In various embodiments, the method may include administering an effective amount of a non-antibiotic macrolide. Suitable non-antibiotic macrolides include rapamycin and rapamycin derivatives, such as temsirolimus, everolimus, ridaforolimus, and the like. In various embodiments, the methods may include administering an effective amount of at least about 1 mg of a non-antibiotic macrolide, or between about 1 mg to about 40 mg of a non-antibiotic macrolide, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg or any range between any two of these amounts including between about 2 mg to about 30 mg, and between about 6 mg to about 40 mg of a non-antibiotic macrolide once, twice, or three or more times daily for at least 10 days.

In various embodiments, the method may include administering an effective amount of a non-antibiotic macrolide that includes an initial administration of a loading dose followed by a maintenance dose of a non-antibiotic macrolide. Suitable non-antibiotic macrolides include rapamycin and rapamycin derivatives, such as temsirolimus, everolimus, ridaforolimus, and the like. In various embodiments, the method may include administering an effective amount of a non-antibiotic macrolide that includes an initial administration of a loading dose of an effective amount of at least about 2 mg of a non-antibiotic macrolide, or between 2 mg to about 10 mg, or 2, 3, 4, 5, 6, 7, 8, 9, and 10 mg or any range between any two of these amounts including between about 2 mg to about 8 mg, and between about 5 mg to about 7 mg followed by a maintenance dose of an effective amount of a non-antibiotic macrolide of at least about 1 mg of a non-macrolide antibiotic, or between about 1 mg to about 40 mg, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 mg or any range between any two of these amounts including between about 2 mg to about 30 mg, and between about 6 mg to about 40 mg of a non-antibiotic macrolide once, twice, or three or more times daily for at least 5 days and up to 12 weeks.

In various embodiments, the methods may include administering an effective amount of an omega-3 fatty acid derivative in a dosing regimen with an effective amount of a biguanide antihyperglycemic agent.

In various embodiments, the methods may include administering an effective amount of an omega-3 fatty acid derivative in a dosing regimen with an effective amount of a flavonoid.

In various embodiments, the methods may include administering an effective amount of a biguanide antihyperglycemic agent in a dosing regimen with an effective amount of a flavonoid.

In various embodiments, the methods may include administering an effective amount of an omega-3 fatty acid derivative in a dosing regimen with an effective amount of a biguanide antihyperglycemic agent and an effective amount of a flavonoid.

In various embodiments, the methods may include administering an effective amount of a thyroid hormone. Suitable thyroid hormones include the T3 liothyronine. In various embodiments, the methods of the disclosure may include administering an effective amount of at least about 1 μg of liothyronine, or between about 1 μg to about 250 μg, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 μg, or any range between any two of these amounts including about 5 μg to about 150 μg, or about 10 μg to about 100 μg, or about 10 μg to about 25 μg, or about 25 μg to about 150 μg, or about 25 μg to about 250 μg either weekly, bimonthly, or monthly; however, thyroid hormones should not be given daily and administration should not exceed three days per week. In some preferred methods, the administration of thyroid hormones is dependent upon the clinical response and tolerance of the patient and may continue long-term including many years. In some preferred methods, the amount of liothyronine administered is sufficient to maintain an optimum level of liothyronine in the blood of a subject receiving an administration of the composition.

In various embodiments, the methods may include diagnosing thyroid functions in each patient prior to administration of an effective amount of a thyroid hormone. In various embodiments, the methods for patients requiring thyroid hormone replacement in order to establish normal thyroid functions may preferentially be administered a T3 thyroid hormone. In various embodiments, patients with normal thyroid functions may be administered a low dose of a T3 thyroid hormone (e.g. 5 to 10 μg of liothyronine) combined with a high dose of a flavonoid. In various embodiments, the methods may include administering a combination of a low dose of a T3 thyroid hormone and a high dose of a flavonoid that effectively elicits a synergistic effect of increasing fat metabolism and promoting cellular senescence. In various embodiments, other than promoting euthyroid in patients, the methods include administering a thyroid hormone concurrent with high doses of a flavonoid, such as fisetin.

In various embodiments, the methods may include administering an effective amount of a combination of thyroid hormones. Suitable thyroid hormones that may be included in the combination include the T3 liothyronine. In various embodiments, the methods may include administering an effective amount of a combination of one or more thyroid hormones with an effective amount of a flavonoid. In various embodiments, the methods may include administering an effective amount of one or more thyroid hormones and an effective amount of a high dose of a flavonoid, which is associated with a senolytic effect unless the patient is hypo thyroid on the days of the week the thyroid hormone is administered. In various embodiments, the methods may include administering a combination of an effective amount of one or more thyroid hormones with either an effective amount of a flavonoid or a high dose of a flavonoid, wherein only some of the compositions that include an effective amount of a flavonoid also include an effective amount of one or more thyroid hormones. For example, an effective amount of one or more thyroid hormones may be included only in one of two weekly compositions administered that includes an effective amount of a flavonoid or a high dose of a flavonoid, which composition could be either the first or second weekly composition administered that includes a flavonoid or high dose flavonoid.

In various embodiments, the methods may include administering an effective amount of a combination of one or more mTOR inhibitors with an effective amount of one or more thyroid hormones. In various embodiments, the methods may include administering an effective amount of one or more mTOR inhibitors with an effective amount of one or more thyroid hormones and an effective amount of a flavonoid. In various embodiments, the methods may include administering an effective amount of one or more mTOR inhibitors with an effective amount of one or more thyroid hormones and an effective amount of a high dose of a flavonoid, which is associated with a senolytic effect. In various embodiments, the methods may include administering a combination of an effective amount of one or more mTOR inhibitors with an effective amount of one or more thyroid hormones and either an effective amount of a flavonoid or a high dose of a flavonoid, wherein only some of the compositions that include an effective amount of a flavonoid also include an effective amount of one or more thyroid hormones. For example, an effective amount of one or more thyroid hormones may be included only in one of two weekly compositions administered that includes an effective amount of a flavonoid or a high dose of a flavonoid, which composition could be either the first or second weekly composition administered that includes a flavonoid or high dose flavonoid. In various embodiments, the one or more thyroid hormones should be administered for short durations (for example, two days a week, bimonthly, or monthly) during any periods of a dosing regimen that include a high dose of a flavonoid.

In some preferred forms, the methods include administering a macrolide, such as rapamycin; in combination with a biguanide antihyperglycemic agent, such as metformin; in combination with an omega-3 fatty acid derivative, such as icosapent ethyl; in combination with a flavonoid, such as fisetin; and in combination with a T3 thyroid hormone, such as liothyronine.

In various embodiments, the method may include administering an effective amount of a corticosteroid. Suitable corticosteroids include dexamethasone. In various embodiments, the methods may include administering an effective amount of at least about 5 mg of a corticosteroid, or between about 5 mg to about 50 mg, or 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg or any range between any two of these amounts including between about 5 mg to about 40 mg, and about 5 mg to about 20 mg of a corticosteroid as a single dose or as a daily dose administered once, twice, three, or four or more times daily.

In various embodiments, the method may include administering an effective amount of a vitamin D derivative. Suitable vitamin D derivatives include vitamin D3 (cholecalciferol). In various embodiments, the methods may include administering an effective amount of at least about 500 international units (IU) of vitamin D3, or between about 500 IU to about 60000 IU of vitamin D3 daily, or 500, 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, 25000, 25500, 26000, 26500, 27000, 27500, 28000, 28500, 29000, 29500, 30000, 30500, 31000, 31500, 32000, 32500, 33000, 33500, 34000, 34500, 35000, 35500, 36000, 36500, 37000, 37500, 38000, 38500, 39000, 39500, 40000, 40500, 41000, 41500, 42000, 42500, 43000, 43500, 44000, 44500, 45000, 45500, 46000, 46500, 47000, 47500, 48000, 48500, 49000, 49500, 50000, 50500, 51000, 51500, 52000, 52500, 53000, 53500, 54000, 54500, 55000, 55500, 56000, 56500, 57000, 57500, 58000, 58500, 59000, 59500, or 60000 IU or any range between any two of these amounts including between about 2000 IU to about 30000 IU, and about 5000 IU to about 50000 IU of vitamin D3 as a single weekly dose or as a daily dose administered once, twice, three, or four or more times daily. In some preferred forms, the amount of vitamin D3 is sufficient to maintain an optimal level of vitamin D3 in the blood of a subject receiving an administration of the composition.

In various embodiments, the method may include administering an effective amount of one or more of a vitamin B derivative in combination with at least one of vitamin A, vitamin C, vitamin E, vitamin K, calcium, phosphorus, iodine, magnesium, zinc, selenium, copper, manganese, chromium, molybdenum, chloride, and potassium. Suitable vitamin B derivatives include thiamin (B1), riboflavin (B2), niacin (B3), pantothenic acid (B5), pyridoxine (B6), biotin (B7), folate (B9), and cyanocobalamin (B12). In various embodiments, the methods may include administering an effective amount of each component in relation to their percentage of daily value (DV) as recommended by the United States Food and Drug administration (FDA). In various embodiments, the methods may include administering an effective amount of at least one of the vitamins or minerals noted above. In some forms, the amount of each individual vitamin or mineral is at least about 1% to about 5000% DV of each included vitamin B derivative, an effective amount of at least about 1% to about 500% DV of vitamin A, an effective amount of at least about 1% to about 5000% DV of vitamin C, an effective amount of at least about 1% to about 500% DV of vitamin E, an effective amount of at least about 1% to about 500% DV of vitamin K, an effective amount of at least about 1% to about 500% DV of calcium, an effective amount of at least about 1% to about 500% DV of phosphorus, an effective amount of at least about 1% to about 500% DV of iodine, an effective amount of at least about 1% to about 500% DV of magnesium, an effective amount of at least about 1% to about 500% DV of zinc, an effective amount of at least about 1% to about 500% DV of selenium, an effective amount of at least about 1% to about 500% DV of copper, an effective amount of at least about 1% to about 500% DV of manganese, an effective amount of at least about 1% to about 500% DV of chromium, an effective amount of at least about 1% to about 500% DV of molybdenum, an effective amount of at least about 1% to about 500% DV of chloride, and an effective amount of at least about 1% to about 500% DV of potassium once, twice, three, or four or more times daily.

In various embodiments, the methods may include administering an effective amount of an omega-3 fatty acid derivative in a dosing regimen with an effective amount of a biguanide antihyperglycemic agent, an effective amount of a vitamin D derivative and an effective amount of a vitamin and mineral supplement.

In various embodiments, the methods may include administering an effective amount of an omega-3 fatty acid derivative in a dosing regimen with an effective amount of a flavonoid, an effective amount of a vitamin D derivative and an effective amount of a vitamin and mineral supplement.

In various embodiments, the methods may include administering an effective amount of an omega-3 fatty acid derivative in a dosing regimen with an effective amount of a non-antibiotic macrolide, an effective amount of a vitamin D derivative and an effective amount of a vitamin and mineral supplement.

In various embodiments, the methods may include administering an effective amount of an omega-3 fatty acid derivative in a dosing regimen with an effective amount of a biguanide antihyperglycemic agent, an effective amount of a flavonoid, an effective amount of a vitamin D derivative and an effective amount of a vitamin and mineral supplement.

In various embodiments, the methods may include administering an effective amount of an omega-3 fatty acid derivative in a dosing regimen with an effective amount of a biguanide antihyperglycemic agent, an effective amount of a non-antibiotic macrolide, an effective amount of a vitamin D derivative and an effective amount of a vitamin and mineral supplement.

In various embodiments, the methods may include administering an effective amount of an omega-3 fatty acid derivative in a dosing regimen with an effective amount of a flavonoid, an effective amount of a non-antibiotic macrolide, an effective amount of a vitamin D derivative and an effective amount of a vitamin and mineral supplement.

In various embodiments, the methods may include administering an effective amount of an omega-3 fatty acid derivative in a dosing regimen with an effective amount of a biguanide antihyperglycemic agent, an effective amount of a flavonoid, an effective amount of a non-antibiotic macrolide, an effective amount of a vitamin D derivative and an effective amount of a vitamin and mineral supplement.

In various embodiments, the methods may include administering an effective amount of a biguanide antihyperglycemic agent in a dosing regimen with an effective amount of a flavonoid, an effective amount of a vitamin D derivative and an effective amount of a vitamin and mineral supplement.

In various embodiments, the methods may include administering an effective amount of a biguanide antihyperglycemic agent in a dosing regimen with an effective amount of a non-antibiotic macrolide, an effective amount of a vitamin D derivative and an effective amount of a vitamin and mineral supplement.

In various embodiments, the method may include administering an effective amount of a biguanide antihyperglycemic agent in a dosing regimen with an effective amount of a flavonoid, an effective amount of a non-antibiotic macrolide, an effective amount of a vitamin D derivative and an effective amount of a vitamin and mineral supplement.

In various embodiments, the method may include administering an effective amount of a non-antibiotic macrolide in a dosing regimen with an effective amount of a corticosteroid.

In various embodiments, the methods may include administering the effective amount of the compositions of the disclosure that may further contain additional pharmaceutically acceptable carriers, excipients, other medicinal or pharmaceutical agents, carriers, adjuvants, etc.

In various embodiments, the methods may include administering an effective amount of the compositions of the disclosure to a patient through any suitable route of administration effective in delivering an amount of active agent or active agents to a patient. Suitable routes of administration include oral, intravascular, intramuscular, subcutaneous, parenteral, enteral, and rectal or the like.

In various embodiments, the compositions may be administered by emergency healthcare professionals, first responders and soldiers away from a traditional treatment center, orally or intravenously, as soon as possible following diagnosis of ABI and/or PTSD.

In various embodiments, the methods may include administering the effective amount of the compositions comprised of each of the ingredients in a single administration form, such as a pill, tablet, capsule, oral solution, injection solution, infusion solution, or any of the forms described herein. In various embodiments, the methods may include administering an effective amount of the compositions of the disclosure from a kit comprising each of the individual ingredients, together with instructions for administering each ingredient. In some forms of the kit, certain ingredients will already be combined such that one, two, three, four, or more of the components or ingredients of the composition are in a single administration form as described herein.

This written description uses examples to disclose the subject matter herein, including the best mode, and also to enable any person skilled in the art to practice the subject matter disclosed herein, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A pharmaceutical composition for the treatment of one or more acquired brain injuries, and/or post-traumatic stress disorder, and or a degenerative neurological disorder comprising an effective amount of one or more mTOR inhibitors.

2. The pharmaceutical composition of claim 1, wherein the one or more mTOR inhibitors is selected from the group consisting of: an omega-3 fatty acid derivative, a biguanide antihyperglycemic agent, a flavonoid, a non-antibiotic macrolide, and combinations thereof.

3. The pharmaceutical composition of claim 2, wherein the omega-3 fatty acid derivative is icosapent ethyl.

4. The pharmaceutical composition of claim 2, wherein the biguanide antihyperglycemic agent is metformin.

5. The pharmaceutical composition of claim 2, wherein the flavonoid is selected from the group consisting of fisetin and fisetin derivatives.

6. The pharmaceutical composition of claim 2, wherein the non-antibiotic macrolide is selected from the group consisting of rapamycin and rapamycin derivatives.

7. The pharmaceutical composition of claim 2, wherein the pharmaceutical composition further comprises an effective amount of one or more thyroid hormones.

8. The pharmaceutical composition of claim 1, wherein the pharmaceutical composition further comprises a corticosteroid.

9. The pharmaceutical composition of claim 1, wherein the pharmaceutical composition further comprises an effective amount of one or more additional active ingredients selected from the group consisting of: a vitamin D derivative, vitamin A, a vitamin B derivative, folate, vitamin C, vitamin E, vitamin K, thiamin, riboflavin, niacin, biotin, pantothenic acid, calcium, phosphorous, iodine, magnesium, zinc, selenium, copper, manganese, chromium, molybdenum, chloride, potassium and combinations thereof.

10. A method of treating, preventing, reducing the incidence of, or reducing the severity of one or more acquired brain injuries, PTSD, or a degenerative neurological disorder comprising administering an effective amount of the pharmaceutical composition of claim 1.

11. A method of treating, preventing, reducing the incidence of, or reducing the severity of post-traumatic stress disorder comprising administering an effective amount of the pharmaceutical composition of claim 1.

12. The composition of claim 1, further comprising an effective amount of one or more thyroid hormones.

13. The composition of claim 1, wherein the pharmaceutical composition further comprises a corticosteroid.
